SEQUENCE LISTING (26 sequences)

SEQ ID NO: 1 - DNA, 21 bp, Artificial Sequence
  p16 forward primer
gatccaggtgggtagaaggtc

SEQ ID NO: 2 - DNA, 19 bp, Artificial Sequence
  p16 reverse primer
cccctgcaaacttcgtcct

SEQ ID NO: 3 - DNA, 22 bp, Artificial Sequence
  p21 forward primer
aggtggacctggagactctcag

SEQ ID NO: 4 - DNA, 22 bp, Artificial Sequence
  p21 reverse primer
tcctcttggagaagatcagccg

SEQ ID NO: 5 - DNA, 20 bp, Artificial Sequence
  GAPDH forward primer
acccactcctccacctttga

SEQ ID NO: 6 - DNA, 22 bp, Artificial Sequence
  GAPDH reverse primer
ctgttgctgtagccaaattcgt

SEQ ID NO: 7 - DNA, 21 bp, Artificial Sequence
  IL-6 forward primer
ccacacagacagccactcacc

SEQ ID NO: 8 - DNA, 22 bp, Artificial Sequence
  IL-6 reverse primer
ctacatttgccgaagagccctc

SEQ ID NO: 9 - DNA, 23 bp, Artificial Sequence
  IL-8 forward primer
ctctcttggcagccttcctgatt

SEQ ID NO: 10 - DNA, 23 bp, Artificial Sequence
  IL-8 reverse primer
aacttctccacaaccctctgcac

SEQ ID NO: 11 - DNA, 24 bp, Artificial Sequence
  MMP-1 forward primer
agctagctcaggatgacattgatg

SEQ ID NO: 12 - DNA, 17 bp, Artificial Sequence
  MMP-1 reverse primer
gccgatgggctggacag

SEQ ID NO: 13 - DNA, 21 bp, Artificial Sequence
  IL-1B forward primer
cagctacgaatctccgaccac

SEQ ID NO: 14 - DNA, 20 bp, Artificial Sequence
  IL-1B reverse primer
ggcagggaaccagcatcttc

SEQ ID NO: 15 - DNA, 22 bp, Artificial Sequence
  TNF-alpha forward primer
atgagcactgaaagcatgatcc

SEQ ID NO: 16 - DNA, 22 bp, Artificial Sequence
  TNF-alpha reverse primer
gagggctgattagagagaggtc

SEQ ID NO: 17 - DNA, 21 bp, Artificial Sequence
  TGF-beta1 forward primer
agggctaccatgccaacttct

SEQ ID NO: 18 - DNA, 21 bp, Artificial Sequence
  TGF-beta1 reverse primer
ccgggttatgctggttgtaca

SEQ ID NO: 19 - DNA, 23 bp, Artificial Sequence
  GM-CSF forward primer
cactgctgctgagatgaatgaaa

SEQ ID NO: 20 - DNA, 20 bp, Artificial Sequence
  GM-CSF reverse primer
gtctgtaggcaggtcggctc

SEQ ID NO: 21 - DNA, 20 bp, Artificial Sequence
  CXCL1 forward primer
gaaagcttgcctcaatcctg

SEQ ID NO: 22 - DNA, 20 bp, Artificial Sequence
  CXCL1 reverse primer
cttcctcctcccttctggtc

SEQ ID NO: 23 - DNA, 22 bp, Artificial Sequence
  CCL2 forward primer
gagaggctgagactaacccaga

SEQ ID NO: 24 - DNA, 22 bp, Artificial Sequence
  CCL2 reverse primer
atcacagcttctttgggacact

SEQ ID NO: 25 - DNA, 20 bp, Artificial Sequence
  CCL3 forward primer
ggctctctgcaaccagttct

SEQ ID NO: 26 - DNA, 21 bp, Artificial Sequence
  CCL3 reverse primer
tgaaattctgtggaatctgcc

(19) United States
(12) Patent Application Publication
(10) Pub. No.: US 2021/0244972 A1
(43) Pub. Date: Aug. 12, 2021

(54) DEVICE FOR REMOVING SENESCENT CELLS COMPRISING ULTRASOUND OUTPUT UNIT

(71) Applicant: KOREA INSTITUTE OF SCIENCE AND TECHNOLOGY, Seoul (KR)

(72) Inventors: So Yeon KIM, Seoul (KR); Hyung Min KIM, Seoul (KR); Ki Joo PAHK, Seoul (KR); In Woo KIM, Seoul (KR); Su Hyun LEE, Seoul (KR)

(73) Assignee: KOREA INSTITUTE OF SCIENCE AND TECHNOLOGY, Seoul (KR)

(21) Appl. No.: 17/103,225

(22) Filed: Nov. 24, 2020

(30) Foreign Application Priority Data
Feb. 10, 2020 (KR) 10-2020-0015524

(51) Int. Cl. A61N 7/00

(52) U.S. Cl. CPC A61N 7/00; A61N 2007/0034

(57) ABSTRACT

The present disclosure relates to a device capable of removing senescent cells by facilitating the phagocytosis of the senescent cells by specifically stimulating the senescent cells using an ultrasound output unit, a kit for removing senescent cells and a method for specifically removing senescent cells, and by selectively and specifically stimulating senescent cells by irradiating ultrasound under a specific condition, thereby promoting the secretion of various cytokines recruiting immune cells, only the senescent cells can be removed specifically and, furthermore, cell regeneration can be promoted.

### CROSS-REFERENCE TO RELATED APPLICATION

This application claims priority to Korean Patent Application No. 10-2020-0015524, filed on Feb. 10, 2020, and all the benefits accruing therefrom under 35 U.S.C. § 119, the contents of which in its entirety are herein incorporated by reference.

### REFERENCE TO SEQUENCE LISTING SUBMITTED VIA EFS-WEB

This application includes an electronically submitted sequence listing in .txt format. The .txt file contains a sequence listing entitled “2021-02-26_5398-0133PUS1_ST25.txt” created on Feb. 26, 2021 and is 5,380 bytes in size. The sequence listing contained in this .txt file is part of the specification and is hereby incorporated by reference herein in its entirety.

### BACKGROUND

### 1. Field

Disclosed in the present disclosure is a device capable of removing senescent cells by specifically stimulating senescent cells using an ultrasound output unit, thereby facilitating phagocytosis of senescent cells, a kit for promoting cell regeneration and a method for specifically removing senescent cells.

### 2. Description of the Related Art

Cellular senescence is a state of permanent cell cycle arrest in response to a variety of cellular stresses such as oxidative stress, DNA damage, and oncogene activation. Cell cycle arrest is a major characteristic of senescent cells and is regulated by induction of p53 (a tumor suppressor) and p21^{Waf1/Cip1} and p16^{Ink4a} cyclin-dependent kinase inhibitors. Senescent cells not only undergo stable cell cycle arrest, but also exhibit morphological changes, including a flat cellular morphology, and upregulation of senescence-associated β-galactosidase (SA-β-gal) activity. In particular, senescent cells display the senescence-associated secretory phenotype (SASP), which is various secretory proteins such as chemokines, pro-inflammatory cytokines, growth factors, and proteases. The SASP dynamically changes over time, has distinct features depending on the senescence state, and can elicit beneficial or deleterious effects according to the context.

Although senescent cells contribute to embryonic development, wound healing, and cellular reprogramming, they were recently recognized to be the major cause of aging and age-related diseases, such as idiopathic pulmonary fibrosis (IPF), fatty liver, liver cirrhosis, arteriosclerosis, diabetes, and arthritis, and therefore various approaches to remove these cells have been developed. The SASP plays important roles in immune surveillance, and immune cell-mediated clearance of senescent cells has been proposed. The SASP is a potent attractant to recruit immune cells, such as macrophages, neutrophils, T lymphocytes, and natural killer cells, which eliminate senescent cells. Therefore, facilitation of immune cell-dependent elimination of senescent cells via precise control of the SASP can be utilized to treat various diseases caused by senescent cells.

Recently, it has been reported that the removal of senescent cells can help to prolong one's life or defeat aging, as well as treat age-related diseases. For example, improved therapeutic efficacy in animal model has been reported when senescent cells are actively removed by using cell-specific biomarker p16^{INK4a} as a promoter, or a drug capable of selectively killing senescent cells, age related diseases. It has been found out the selective removal of senescent cells in an animal model not only prolongs average life span but also leads to remarkable change in aging phenotypes such as hair gloss, eye clearness, skin gloss, body size, etc. Meanwhile, drugs that selectively remove senescent cells are called as senolytic drugs. Typical examples include D+Q (dasatinib+quercetin), UBX0101, etc. It has been demonstrated that senolytic drugs can prolong life span, reduce senescence-associated symptoms and treat osteoarthritis in animal models. According to a clinical report published in 2019, when the senolytic drug D+Q was treated to patients with IPF, the walking time, chair stand time of the patients was significantly increased. Because of these possible therapeutic implication, tremendous efforts to develop the senolytic drugs as therapeutic agents have been reported. Oisin (USA) and Cleara Biotech (the Netherlands) are attempting to treat aging-associated diseases and cancers by selectively removing senescent cells. Recently, efforts to develop senolytic drugs are also reported in Korea. However, the proposed approaches are mostly based on chemical drugs or biochemical methods and no other strategy has been developed.

On the other hand, ultrasound is a promising non-invasive tool for diagnosis and therapy. In therapeutic applications, ultrasound can be classified as high intensity or low intensity. The high-intensity ultrasound mainly utilizes thermal ablation effect and is used for treatment of patients with uterine fibroid, prostatic hyperplasia, prostate cancer, metastatic bone tumor and hand tremor in clinical applications. In addition, clinical therapies using the high-intensity ultrasound, e.g., extracorporeal shockwave therapy, are performed for treatment of bone diseases and alleviation of joint pain. The low-intensity ultrasound is applicable to treatment of neurological/psychiatric disorders. Furthermore, it has been reported that ultrasound can regulates secretion of cytokines associated with inflammation and promotes wound healing and bone repair. In particular, ultrasound treatment can alleviate pain in arthritis by inhibiting the secretion of inflammatory cytokines. Here, the inventors have developed a method specifically and selectively stimulated senescent cells only using ultrasound and have investigated whether immune cells or macrophages can be further recruited and whether the effect of removing senescent cells can be promoted.

### REFERENCES

### Non-Patent Documents

(Non-patent document 1) Low-intensity pulsed ultrasound promotes bone morphogenic protein 9-induced osteogenesis and suppresses inhibitory effects of inflammatory cytokines on cellular responses via Rho-associated kinase 1 in human periodontal ligament fibroblasts, Joji Kusuyama et al., J. Cell Biochem., 2019.
(Non-patent document 2) Low-intensity pulsed ultrasound promotes spinal fusion by regulating macrophage polarization, Zi-Cheng Zhang et al., Biomedicine & Pharmacotherapy, Volume 120, December 2019.

### Patent Documents

(Patent document 1) KR 10-2016-0001890 A.
(Patent document 2) KR 10-2068724 B.

### DISCLOSURE

### Technical Problem

In an aspect, the present disclosure is directed to providing a device for removing senescent cells, which includes an ultrasound output unit, wherein ultrasound outputted from the ultrasound output unit facilities phagocytosis of senescent cells by specifically stimulating the senescent cells.

In another aspect, the present disclosure is directed to providing a kit for removing senescent cells, which includes an ultrasound output unit and an instruction, wherein the instruction describes that ultrasound outputted from the ultrasound output unit facilities phagocytosis of senescent cells by specifically stimulating the senescent cells.

In another aspect, the present disclosure is directed to providing a kit for promoting cell regeneration, which includes an ultrasound output unit and an instruction, wherein the instruction describes that ultrasound outputted from the ultrasound output unit facilities phagocytosis of senescent cells and cell regeneration by specifically stimulating the senescent cells and that the cell regeneration is caused by migration of normal cells facilitated by the ultrasound outputted from the ultrasound output unit.

In another aspect, the present disclosure is directed to providing a method for specifically removing senescent cells, which includes irradiating ultrasound to skin surface of a subject in need of removal of senescent cells, wherein the ultrasound irradiated to the skin surface facilities phagocytosis of senescent cells by specifically stimulating the senescent cells among skin cells.

### Technical Solution

In an aspect, the present disclosure provides a device for removing senescent cells, which includes an ultrasound output unit, wherein ultrasound outputted from the ultrasound output unit facilities phagocytosis of senescent cells by specifically stimulating the senescent cells.

In another aspect, the present disclosure provides a kit for removing senescent cells, which includes an ultrasound output unit and an instruction, wherein the instruction describes that ultrasound outputted from the ultrasound output unit facilities phagocytosis of senescent cells by specifically stimulating the senescent cells.

In another aspect, the present disclosure provides a kit for promoting cell regeneration, which includes an ultrasound output unit and an instruction, wherein the instruction describes that ultrasound outputted from the ultrasound output unit facilities phagocytosis of senescent cells and cell regeneration by specifically stimulating the senescent cells and that the cell regeneration is caused by migration of normal cells facilitated by the ultrasound outputted from the ultrasound output unit.

In another aspect, the present disclosure provides a method for specifically removing senescent cells, which includes irradiating ultrasound to skin surface of a subject in need of removal of senescent cells, wherein the ultrasound irradiated to the skin surface facilities phagocytosis of senescent cells by specifically stimulating the senescent cells among skin cells.

### Advantageous Effects

According to an aspect of the present disclosure, by selectively and specifically stimulating senescent cells by irradiating ultrasound under a specific condition, thereby promoting the secretion of various cytokines recruiting immune cells, only the senescent cells can be specifically removed and, furthermore, cell regeneration can be promoted. Accordingly, the present disclosure is applicable to treatment of aging-associated diseases caused by accumulated senescent cells and may be usefully utilizing in a device for skin regeneration and antiaging by applying ultrasound stimulation.

### DESCRIPTION OF DRAWINGS

FIG. 1 schematically illustrates the effect of ultrasound irradiation on senescent cell using a device according to an aspect of the present disclosure and an effect derived therefrom.

FIG. 2A and FIG. 2B illustrate senescent cell stimulation experiment by ultrasound irradiation using a device according to an aspect of the present disclosure.

Specifically, FIG. 2A shows images, as an example of ultrasound irradiation experiment, irradiation of ultrasound to a glass plate-based culture dish with cells (including fibroblasts of normal skin or fibroblasts of aged skin) and the culture dish was placed on an ultrasound transducer. And, FIG. 2B shows an experimental schedule of ultrasound irradiation to dermal fibroblasts using a device according to an aspect of the present disclosure.

FIG. 3 shows a measurement data of cell viability upon ultrasound irradiation using a device according to an aspect of the present disclosure. Specifically, the change in the cell viability of fibroblasts of normal skin (young) and fibroblasts of aged skin (old) was measured before (con) and after (+US) ultrasound irradiation.

FIG. 4A shows a cell proliferation rate upon ultrasound irradiation using a device according to an aspect of the present disclosure. Specifically, the cell proliferation rate of fibroblasts of normal skin (young) and fibroblasts of aged skin (old) was measured before (con) and after (+US) ultrasound irradiation.

FIG. 4B and FIG. 4C show qRT-PCR measurement of cell cycle-related genes and cell cycle analysis upon ultrasound irradiation using a device according to an aspect of the present disclosure. Specifically, FIG. 4B show the relative mRNA levels of p16 and p21, which are cell cycle-related proteins, in fibroblasts of normal skin (young) and fibroblasts of aged skin (old) before (−US) and after (+US) ultrasound irradiation by qRT-PCR. And, FIG. 4C show a FACS data of fibroblasts of normal skin (young) and fibroblasts of aged skin (old) before (−US) and after (+US) ultrasound irradiation by DNA staining with Pl.

FIG. 5 shows optical images representing the activity of senescence-associated β-galactosidase (SA β-gal), which is a senescent cell-specific protein, in fibroblasts of normal skin (young) and fibroblasts of aged skin (old) before (con) and after (+US) ultrasound irradiation using a device according to an aspect of the present disclosure by X-gal staining.

FIG. 6 shows relative mRNA levels of senescence-associated secretory phenotypes (SASP) including IL-6, IL-8, MMP-1 and IL-1B, which are senescent cell-specific cytokines, in fibroblasts of normal skin (young) and fibroblasts of aged skin (old) before (con) and after (+US) ultrasound irradiation using a device according to an aspect of the present disclosure by qRT-PCR.

FIG. 7A schematically illustrates transwell migration experiment for measuring increased migration or recruiting effect of senescent cell-specific monocytes (THP-1) and macrophages (M0, M1, M2) by ultrasound irradiation using a device according to an aspect of the present disclosure.

FIGS. 7B-7D show optical images of staining monocytes and macrophages with crystal violet in order to investigate the change in senescent cell-dependent migration of monocytes (THP-1) and macrophages (M0, M1, M2) by ultrasound irradiation using a device according to an aspect of the present disclosure. FIG. 7B shows images obtained by staining the migrated monocytes and macrophage, FIG. 7C shows the degree of migrated monocytes measured by the optical images of stained monocytes, and FIG. 7D shows the degree of migrated macrophages (M0, M1, M2) measured by the optical images of stained macrophages.

FIG. 8 shows a relative mRNA levels of cytokines involved in immune cells recruiting in fibroblasts of normal skin (young) and fibroblasts of aged skin (old) by qRT-PCR before (con) and after (+US) ultrasound irradiation using a device according to an aspect of the present disclosure.

FIG. 9A and FIG. 9B show a cell migration assay data of normal fibroblasts with the culture media from fibroblasts of normal skin (young) and fibroblasts of aged skin (old) before (con) and after (US) ultrasound irradiation using a device according to an aspect of the present disclosure.

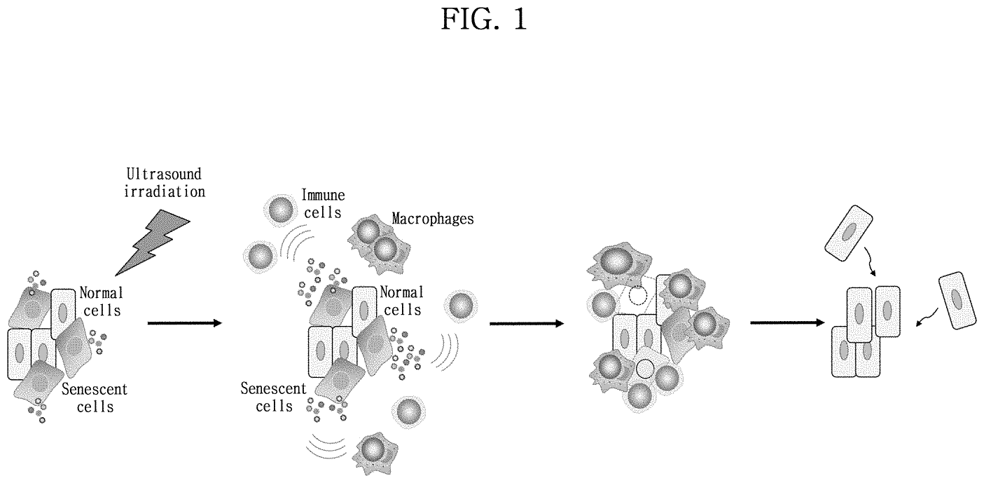

### BEST MODE

Hereinafter, the present disclosure is described in detail referring to the attached drawings which illustrate specific exemplary embodiments of the present disclosure. The exemplary embodiments will be described in detail such that those skilled in the art can easily carry out the present disclosure. It is to be understood that, although the various exemplary embodiments of the present disclosure are different from each other, they are not necessarily mutually exclusive. For example, the particular shape, structure and feature described relating to an exemplary embodiment may be embodied in other exemplary embodiments without departing from the spirit and scope of the present disclosure. In addition, it is to be understood that the location or arrangement of each component described in each exemplary embodiment may be changed without departing from the spirit and scope of the present disclosure. Accordingly, the following description is not intended to limit the scope of the present disclosure, and the scope of the present disclosure will be limited only by the appended claims.

In an aspect, the term “specific” used in the present disclosure has the same meaning as the term “selective” and may be used interchangeably with each other.

In an aspect, the present disclosure provides a device for removing senescent cells, which includes an ultrasound output unit, wherein ultrasound outputted from the ultrasound output unit facilities phagocytosis of senescent cells by specifically stimulating the senescent cells.

In an aspect, the present disclosure may provide a device for removing senescent cells, which includes an ultrasound output unit.

In an aspect of the present disclosure, the ultrasound output unit is a device capable of outputting or irradiating ultrasound. Specifically, it may be an ultrasound transducer, but is not limited in the type or components of the ultrasound output unit as long as it can irradiate ultrasound to a subject.

In an aspect of the present disclosure, the ultrasound outputted from the ultrasound output unit may be low-intensity ultrasound with a center frequency of 5 kHz to 450 MHz. Specifically, the center frequency of the ultrasound outputted from the ultrasound output unit may be 5 kHz or higher, 10 kHz or higher, 50 kHz or higher, 100 kHz or higher, 150 kHz or higher, 200 kHz or higher, 250 kHz or higher, 300 kHz or higher, 350 kHz or higher, 400 kHz or higher, 450 kHz or higher, 500 kHz or higher, 550 kHz or higher, 600 kHz or higher, 650 kHz or higher, 700 kHz or higher, 750 kHz or higher, 800 kHz or higher, 850 kHz or higher, 900 kHz or higher, 910 kHz or higher, 920 kHz or higher, 930 kHz or higher, 940 kHz or higher, 950 kHz or higher, 960 kHz or higher, 970 kHz or higher, 980 kHz or higher, 990 kHz or higher, 1 MHz or higher, 1.1 MHz or higher, 1.2 MHz or higher, 1.3 MHz or higher, 1.4 MHz or higher, 1.5 MHz or higher, 1.6 MHz or higher, 1.7 MHz or higher, 1.8 MHz or higher, 1.9 MHz or higher, 2 MHz or higher, 2.1 MHz or higher, 2.2 MHz or higher, 2.4 MHz or higher, 2.5 MHz or higher, 2.6 MHz or higher, 2.7 MHz or higher, 2.8 MHz or higher, 2.9 MHz or higher, 3 MHz or higher, 4 MHz or higher, 5 MHz or higher, 10 MHz or higher, 50 MHz or higher, 100 MHz or higher, 150 MHz or higher, 200 MHz or higher, 250 MHz or higher, 300 MHz or higher, 350 MHz or higher, 400 MHz or higher, and may be 450 MHz or lower, 400 MHz or lower, 350 MHz or lower, 300 MHz or lower, 250 MHz or lower, 200 MHz or lower, 150 MHz or lower, 100 MHz or lower, 50 MHz or lower, 10 MHz or lower, 5 MHz or lower, 4 MHz or lower, 3 MHz or lower, 2.9 MHz or lower, 2.8 MHz or lower, 2.7 MHz or lower, 2.6 MHz or lower, 2.5 MHz or lower, 2.4 MHz or lower, 2.3 MHz or lower, 2.2 MHz or lower, 2.1 MHz or lower, 2 MHz or lower, 1.9 MHz or lower, 1.8 MHz or lower, 1.7 MHz or lower, 1.6 MHz or lower, 1.5 MHz or lower, 1.4 MHz or lower, 1.3 MHz or lower, 1.2 MHz or lower, 1.1 MHz or lower, 1 MHz or lower, 990 kHz or lower, 980 kHz or lower, 970 kHz or lower, 960 kHz or lower, 950 kHz or lower, 940 kHz or lower, 930 kHz or lower, 920 kHz or lower, 910 kHz or lower, 900 kHz or lower, 850 kHz or lower, 800 kHz or lower, 750 kHz or lower, 700 kHz or lower, 650 kHz or lower, 600 kHz or lower, 550 kHz or lower, 500 kHz or lower, 450 kHz or lower, 400 kHz or lower, 350 kHz or lower, 300 kHz or lower, 250 kHz or lower, 200 kHz or lower, 150 kHz or lower, 100 kHz or lower, 50 kHz or lower or 10 kHz or lower, more specifically 15 kHz or higher and 150 MHz or lower, further more specifically 150 kHz or higher and 15 MHz or lower. However, the center frequency of the ultrasound may be controlled depending on the subject to which the ultrasound is irradiated, the part of the subject, the type of cells or the purpose of the ultrasound irradiation, and is not limited to the range described above.

In an aspect of the present disclosure, the ultrasound outputted from the ultrasound output unit may have a sound pressure of −10 MPa to 10 MPa. Specifically, the sound pressure of the ultrasound outputted from the ultrasound output unit may be −10 MPa or higher, −8 MPa or higher, −6 MPa or higher, −4 MPa or higher, −2 MPa or higher, −1 MPa or higher, −800 kPa or higher, −600 kPa or higher, −400 kPa or higher, −200 kPa or higher, −100 kPa or higher, −80 kPa or higher, −60 kPa or higher, −40 kPa or higher, −20 kPa or higher, 0 kPa or higher, 20 kPa or higher, 40 kPa or higher, 60 kPa or higher, 80 kPa or higher, 100 kPa or higher, 120 kPa or higher, 140 kPa or higher, 160 kPa or higher, 180 kPa or higher, 200 kPa or higher, 210 kPa or higher, 220 kPa or higher, 230 kPa or higher, 240 kPa or higher, 260 kPa or higher, 280 kPa or higher, 300 kPa or higher, 500 kPa or higher, 1 MPa or higher, 2 MPa or higher, 3 MPa or higher, 4 MPa or higher, 5 MPa or higher, 6 MPa or higher, 7 MPa or higher, 8 MPa or higher or 9 MPa or higher, and may be 10 MPa or lower, 9 MPa or lower, 8 MPa or lower, 7 MPa or lower, 6 MPa or lower, 5 MPa or lower, 4 MPa or lower, 3 MPa or lower, 2 MPa or lower, 1 MPa or lower, 900 kPa or lower, 800 kPa or lower, 700 kPa or lower, 600 kPa or lower, 500 kPa or lower, 400 kPa or lower, 300 kPa or lower, 280 kPa or lower, 260 kPa or lower, 250 kPa or lower, 240 kPa or lower, 230 kPa or lower, 220 kPa or lower, 200 kPa or lower, 150 kPa or lower, 100 kPa or lower, 50 kPa or lower, 0 kPa or lower, −50 kPa or lower, −100 kPa or lower, −500 kPa or lower, −1 MPa or lower, −5 MPa or lower or −10 MPa or lower. However, the sound pressure of the ultrasound may be controlled depending on the subject to which the ultrasound is irradiated, the part of the subject, the type of cells or the purpose of the ultrasound irradiation, and is not limited to the range described above.

In an aspect of the present disclosure, the ultrasound outputted from the ultrasound output unit may have a pulse repetition frequency of 0.02 Hz to 500 kHz. Specifically, the pulse repetition frequency of the ultrasound outputted from the ultrasound output unit may be 0.02 Hz or higher, 0.1 Hz or higher, 1 Hz or higher, 10 Hz or higher, 50 Hz or higher, 60 Hz or higher, 70 Hz or higher, 75 Hz or higher, 80 Hz or higher, 85 Hz or higher, 90 Hz or higher, 91 Hz or higher, 92 Hz or higher, 93 Hz or higher, 94 Hz or higher, 95 Hz or higher, 96 Hz or higher, 97 Hz or higher, 98 Hz or higher, 99 Hz or higher, 100 Hz or higher, 101 Hz or higher, 102 Hz or higher, 103 Hz or higher, 104 Hz or higher, 105 Hz or higher, 106 Hz or higher, 107 Hz or higher, 108 Hz or higher, 109 Hz or higher, 110 Hz or higher, 150 Hz or higher, 200 Hz or higher, 300 Hz or higher, 400 Hz or higher, 500 Hz or higher, 600 Hz or higher, 700 Hz or higher, 800 Hz or higher, 900 Hz or higher, 1 kHz or higher, 10 kHz or higher, 100 kHz or higher, 200 kHz or higher, 300 kHz or higher or 400 kHz or higher, and may be 500 kHz or lower, 400 kHz or lower, 300 kHz or lower, 200 kHz or lower, 100 kHz or lower, 10 kHz or lower, 1 kHz or lower, 900 Hz or lower, 800 Hz or lower, 700 Hz or lower, 600 Hz or lower, 500 Hz or lower, 400 Hz or lower, 300 Hz or lower, 200 Hz or lower, 150 Hz or lower, 140 Hz or lower, 130 Hz or lower, 120 Hz or lower, 110 Hz or lower, 109 Hz or lower, 108 Hz or lower, 107 Hz or lower, 106 Hz or lower, 105 Hz or lower, 104 Hz or lower, 103 Hz or lower, 102 Hz or lower, 101 Hz or lower, 100 Hz or lower, 99 Hz or lower, 98 Hz or lower, 97 Hz or lower, 96 Hz or lower, 95 Hz or lower, 94 Hz or lower, 93 Hz or lower, 92 Hz or lower, 91 Hz or lower, 90 Hz or lower, 85 Hz or lower, 80 Hz or lower, 75 Hz or lower, 70 Hz or lower, 60 Hz or lower, 50 Hz or lower, 40 Hz or lower, 30 Hz or lower, 20 Hz or lower, 10 Hz or lower, 1 Hz or lower or 0.1 Hz or lower, more specifically 0.2 Hz or higher and 50 kHz or lower, further more specifically 2 Hz or higher and 5 kHz or lower. However, the pulse repetition frequency of the ultrasound may be controlled depending on the subject to which the ultrasound is irradiated, the part of the subject, the type of cells or the purpose of the ultrasound irradiation, and is not limited to the range described above.

In an aspect of the present disclosure, the ultrasound outputted from the ultrasound output unit may have a duty cycle of 0.1-99.9%. Specifically, the duty cycle of the ultrasound outputted from the ultrasound output unit may be 0.1% or higher, 1% or higher, 5% or higher, 10% or higher, 11% or higher, 12% or higher, 13% or higher, 14% or higher, 15% or higher, 16% or higher, 17% or higher, 18% or higher, 19% or higher, 20% or higher, 25% or higher, 30% or higher, 35% or higher, 40% or higher, 45% or higher, 50% or higher, 60% or higher, 70% or higher, 80% or higher or 90% or higher, and may be 99% or lower, 90% or lower, 80% or lower, 70% or lower, 60% or lower, 50% or lower, 45% or lower, 40% or lower, 35% or lower, 30% or lower, 29% or lower, 28% or lower, 27% or lower, 26% or lower, 25% or lower, 24% or lower, 23% or lower, 22% or lower, 21% or lower, 20% or lower, 15% or lower, 10% or lower, 5% or lower or 1% or lower, more specifically 1% or higher and 90% or lower, further more specifically 5% or higher and 80% or lower. However, the duty cycle of the ultrasound may be controlled depending on the subject to which the ultrasound is irradiated, the part of the subject, the type of cells or the purpose of the ultrasound irradiation, and is not limited to the range described above.

The device for removing senescent cells according to an aspect of the present disclosure may further include an ultrasound control unit.

In an aspect of the present disclosure, the ultrasound control unit may control and optimize one or more condition selected from a group consisting of the center frequency, sound pressure, pulse repetition frequency and duty cycle of the ultrasound depending on the subject to which the ultrasound is irradiated, the part of the subject, the type of cells or the purpose of the ultrasound irradiation.

In an aspect, the present disclosure may provide a device for removing senescent cells, wherein the ultrasound outputted from the ultrasound control unit specifically stimulates senescent cells.

In an aspect of the present disclosure, the cells may be senescent cells of any subject desired to be removed through ultrasound irradiation, without particular limitation. Specifically, the subject may be a non-human animal such as monkey, dog, cat, rabbit, guinea pig, rat, mouse, cow, sheep, pig, goat, etc. or human, although not being limited thereto. In addition, the subject may be a subject having senescent cells. Specifically, it may be a subject wherein aging phenotypes are expressed due to insufficient removal of senescent cells, more specifically, a subject wherein the activity of senescence-associated β-galactosidase is increased, the expression of a senescent cell-specific cytokine (SASP), e.g., one or more selected from a group consisting of interleukin 6 (IL-6), interleukin 8 (IL-8), matrix metalloproteinase-1 (MMP-1) and interleukin 1β (IL-1β), is increased, or the secretion of senescence-associated secretory phenotype (SASP) is increased, further more specifically, a subject wherein skin aging has progressed, although not being limited thereto.

In an aspect of the present disclosure, the senescent cell refers not only to a cell aged as a result of repeated cell division but also to a cell whose proliferation is impossible or whose function is declined markedly due to the inhibition of cell division due to cell damage caused by various reasons. Specifically, the senescent cell may be a cell aged as a result of repeated cell division, a senescent cell damaged by chemical stimulation such as an anticancer drug, a toxic substance, etc. or physical stimulation such as radiation, UV, etc. or a senescent cell damaged by accumulation of oncogenes, more specifically a cell wherein the activity of senescence-associated β-galactosidase is increased, the expression of a senescent cell-specific cytokine, e.g., one or more selected from a group consisting of interleukin 6 (IL-6), interleukin 8 (IL-8), matrix metalloproteinase-1 (MMP-1) and interleukin 1β (IL-1β), is increased, or the secretion of senescence-associated secretory phenotype (SASP) is increased, although not being limited thereto. In an aspect of the present disclosure, the senescent cell may be one or more selected from a group consisting of a senescent fibroblast, a senescent melanocyte, a senescent keratinocyte, a senescent muscle cell, a senescent epithelial cell, a senescent vascular endothelial cell, a senescent bone cell, a senescent cartilage cell, a senescent cardiac muscle cell, a senescent liver cell, a senescent pancreatic cell and a senescent kidney cell, although not being limited thereto. The senescent cell may be different depending on the subject to which the ultrasound is irradiated, the part of the subject or the purpose of the ultrasound irradiation.

In an aspect of the present disclosure, the stimulation refers to irradiation of ultrasound to cells, specifically cells including senescent cells. Specifically, the stimulation means irradiation of ultrasound to cells to facilitate or inhibit specific reactions in the cells or to affect the phenotype of the cells by facilitating or inhibiting the expression of specific genes or the activity of specific proteins.

In an aspect of the present disclosure, the ultrasound may be irradiated for 1-400 minutes. Specifically, the ultrasound may be irradiated for 1 minute or longer, 2 minutes or longer, 3 minutes or longer, 4 minutes or longer, 5 minutes or longer, 6 minutes or longer, 7 minutes or longer, 8 minutes or longer, 9 minutes or longer, 10 minutes or longer, 11 minutes or longer, 12 minutes or longer, 13 minutes or longer, 14 minutes or longer, 15 minutes or longer, 16 minutes or longer, 17 minutes or longer, 18 minutes or longer, 19 minutes or longer, 20 minutes or longer, 25 minutes or longer, 30 minutes or longer, 35 minutes or longer, 40 minutes or longer, 45 minutes or longer, 50 minutes or longer, 60 minutes or longer, 90 minutes or longer, 120 minutes or longer, 180 minutes or longer, 240 minutes or longer, 300 minutes or longer or 360 minutes or longer, and may be irradiated for 400 minutes or shorter, 360 minutes or shorter, 300 minutes or shorter, 240 minutes or shorter, 180 minutes or shorter, 120 minutes or shorter, 90 minutes or shorter, 60 minutes or shorter, 50 minutes or shorter, 45 minutes or shorter, 40 minutes or shorter, 39 minutes or shorter, 38 minutes or shorter, 37 minutes or shorter, 36 minutes or shorter, 35 minutes or shorter, 34 minutes or shorter, 33 minutes or shorter, 32 minutes or shorter, 31 minutes or shorter, 30 minutes or shorter, 29 minutes or shorter, 28 minutes or shorter, 27 minutes or shorter, 26 minutes or shorter, 25 minutes or shorter, 24 minutes or shorter, 23 minutes or shorter, 22 minutes or shorter, 21 minutes or shorter, 20 minutes or shorter, 15 minutes or shorter, 10 minutes or shorter or 5 minutes or shorter. However, the ultrasound irradiation time may be controlled depending on the subject to which the ultrasound is irradiated, the part of the subject, the type of cells or the purpose of the ultrasound irradiation and is not limited to the range described above.

In an aspect of the present disclosure, the ultrasound may be irradiated 1-10 times a day. Specifically, the ultrasound may be irradiated once, 2 two times or more, 3 two times or more, 4 two times or more, 5 two times or more, 6 two times or more, 7 two times or more, 8 two times or more or 9 two times or more a day, and may be irradiated 10 two times or less, 9 two times or less, 8 two times or less, 7 two times or less, 6 two times or less, 5 two times or less, 4 two times or less, 3 two times or less or 2 two times or less a day. However, the irradiation number of the ultrasound irradiation may be controlled depending on the subject to which the ultrasound is irradiated, the part of the subject, the type of cells or the purpose of the ultrasound irradiation, and is not limited to the range described above.

In an aspect of the present disclosure, the ultrasound may be irradiated with an interval of once a day to once a month. Specifically, it may be irradiated with an interval of once a day, once in 2 days, once in 3 days, once in 4 days, once in 5 days, once in 6 days, once in 7 days, once in 10 days, once in 15 days, once in 20 days, once in 25 days or once in 30 days. However, the interval of the ultrasound irradiation may be controlled depending on the subject to which the ultrasound is irradiated, the part of the subject, the type of cells or the purpose of the ultrasound irradiation, and is not limited to the range described above.

In an aspect of the present disclosure, the senescent cell-specific stimulation means a stimulation which facilitates or inhibits specific reactions specifically in senescent cells as compared to in normal cells or young cells, or leads to a large change in the expression of specific genes or the activity of specific proteins by facilitating or inhibiting the expression of specific genes or the activity of specific proteins, leading to a large change in phenotypes in senescent cells as compared to in normal cells or young cells. Specifically, the senescent cell-specific stimulation may be one which senescent cell-specifically increases the activity of senescence-associated β-galactosidase, or the expression of one or more senescent cell-specific cytokine selected from a group consisting of interleukin 6 (IL-6), interleukin 8 (IL-8), matrix metalloproteinase-1 (MMP-1) and interleukin 1β (IL-1β) specifically in senescent cells as compared to in normal cells or young cells, or increases the migration of monocytes or macrophages in senescent cells as compared to in normal cells or young cells, or increases the expression of one or more immune cell-recruiting cytokine selected from a group consisting of colony-stimulating factor (CSF), chemokine (C-X-C motif) ligand 1 (CXCL1) and chemokine (C-C motif) ligand 3 (CCL3) in senescent cells as compared to normal or young cells, or facilitates the death of senescent cells as compared to normal or young cells, or facilitates cell regeneration, although not being limited thereto.

In an aspect of the present disclosure, the ultrasound outputted from the ultrasound output unit may senescent cell-specifically increase the activity of senescence-associated β-galactosidase (SA β-gal). The senescence-associated β-galactosidase is a hydrolase enzyme which catalyzes the hydrolysis of β-galactosides into monosaccharides only in senescent cells, and is known as a biomarker of cellular senescence along with p16^{INK4a}. In an aspect of the present disclosure, due to the ultrasound outputted from the ultrasound output unit, an increase in the activity of senescence-associated β-galactosidase in senescent cells may be greater than that of normal cells or young cells. Specifically, the ultrasound outputted from the ultrasound output unit may increase the activity of senescence-associated β-galactosidase in senescent cells 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.5 times or more, 3 times or more, 4 times or more, 5 times or more, 6 times or more, 7 times or more, 8 times or more, 9 times or more or 10 times or more as compared to in normal cells or young cells, although not being limited thereto.

In an aspect of the present disclosure, the ultrasound outputted from the ultrasound output unit may senescent cell-specifically increase the expression of one or more senescent cell-specific cytokine selected from a group consisting of interleukin 6 (IL-6), interleukin 8 (IL-8), matrix metalloproteinase-1 (MMP-1) and interleukin 1β (IL-1β). The senescent cell-specific cytokine is also known as senescence-associated secretory phenotype (SASP). Senescent cells expressing the senescence-associated secretory phenotype secrete high levels of inflammatory cytokines, immune modulators, growth factors and proteases. In addition, the senescence-associated secretory phenotype induces chronic inflammation to interrupt normal tissue functions, and stimulates the immune system to remove senescent cells. In an aspect of the present disclosure, the senescent cell-specific cytokine or senescence-associated secretory phenotype may be one or more selected from a group consisting of interleukin 6 (IL-6), interleukin 8 (IL-8), matrix metalloproteinase-1 (MMP-1) and interleukin 1β (IL-1β), although not being limited thereto. In an aspect of the present disclosure, the ultrasound outputted from the ultrasound output unit may increase the expression of the senescent cell-specific cytokine in senescent cells as compared to in normal cells or young cells. Specifically, the ultrasound outputted from the ultrasound output unit may increase the expression of the senescent cell-specific cytokine 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.5 times or more, 3 times or more, 4 times or more, 5 times or more, 6 times or more, 7 times or more, 8 times or more, 9 times or more or 10 times or more as compared to before or without ultrasound irradiation, although not being limited thereto.

In an aspect of the present disclosure, the ultrasound outputted from the ultrasound output unit may facilitate the migration of monocytes or macrophages specifically in senescent cells. The monocytes are white blood cells found in blood vessels, which are capable of phagocytosis. Since the monocytes differentiate into macrophages after migrating to a tissue and perform phagocytosis, increased or activated migration of the monocytes may mean that phagocytosis is facilitated. The macrophages are cells which are distributed in all tissues of a subject, specifically an animal, and play a role in immunity. The macrophages include M0 macrophages, M1 macrophages, M2 macrophages, etc. Among them, M0 macrophages and M1 macrophages are known to play an important role in phagocytosis. In an aspect of the present disclosure, the ultrasound outputted from the ultrasound output unit may facilitate the migration of monocytes or macrophages specifically in senescent cells as compared to in normal cells or young cells. Specifically, the ultrasound outputted from the ultrasound output unit may increase the migration of monocytes or macrophages in senescent cells as compared to in normal cells or young cells 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.5 times or more, 3 times or more, 4 times or more, 5 times or more, 6 times or more, 7 times or more, 8 times or more, 9 times or more or 10 times or more as compared to before or without ultrasound irradiation, although not being limited thereto. In addition, in an aspect of the present disclosure, the ultrasound outputted from the ultrasound output unit may facilitate the migration of M0 macrophages or M1 macrophages specifically in senescent cells as compared to in normal cells or young cells.

In an aspect of the present disclosure, the ultrasound outputted from the ultrasound output unit may senescent cell-specifically increase the expression of one or more immune cell-recruiting cytokine selected from a group consisting of colony-stimulating factor (CSF), chemokine (C-X-C motif) ligand 1 (CXCL1) and chemokine (C-C motif) ligand 3 (CCL3). The immune cell-recruiting cytokine is a cytokine known to recruit macrophages or immune cells. The increased expression of the immune cell-recruiting cytokine may mean facilitated phagocytosis. In an aspect of the present disclosure, the immune cell-recruiting cytokine may be one or more selected from a group consisting of colony-stimulating factor (CSF), chemokine (C-X-C motif) ligand 1 (CXCL1) and chemokine (C-C motif) ligand 3 (CCL3), although not being limited thereto. In addition, in an aspect of the present disclosure, the ultrasound outputted from the ultrasound output unit may increase the expression of the immune cell-recruiting cytokine in senescent cells as compared to in normal cells or young cells 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.5 times or more, 3 times or more, 4 times or more, 5 times or more, 6 times or more, 7 times or more, 8 times or more, 9 times or more or 10 times or more as compared to before or without ultrasound irradiation, although not being limited thereto.

In an aspect of the present disclosure, the ultrasound outputted from the ultrasound output unit may have no senescent cell-specific effect on one or more selected from a group consisting of cell viability, cell proliferation and cell cycle. The absence of the senescent cell-specific effect means that there is no statistically significant difference between the change in cell viability, cell proliferation or cell cycle of normal cells or young cells before (or without) and after ultrasound irradiation and the change in cell viability, cell proliferation or cell cycle of senescent cells before (or without) and after ultrasound irradiation. Therefore, the device of an aspect of the present disclosure can facilitate the death of senescent cells specifically by specifically stimulating senescent cells only and facilitating phagocytosis of senescent cells through ultrasound irradiation without affecting cell viability, cell proliferation or cell cycle.

In an aspect of the present disclosure, the ultrasound outputted from the ultrasound output unit may senescent cell-specifically facilitate the phagocytosis of senescent cells. The phagocytosis refers to a process whereby cell debris, foreign materials, microorganisms, cancer cells, abnormal proteins, etc. are engulfed and degraded. Specifically, it may be phagocytosis by macrophages. In an aspect of the present disclosure, the facilitated phagocytosis of senescent cells may mean facilitated phagocytosis whereby senescent cells are degraded, and the facilitated senescent cell-specific phagocytosis may mean facilitated phagocytosis whereby only senescent cells are removed and killed specifically as compared to normal cells or young cells.

In an aspect of the present disclosure, the ultrasound outputted from the ultrasound output unit may specifically facilitate the death of senescent cells.

In an aspect of the present disclosure, the ultrasound outputted from the ultrasound output unit may facilitate cell regeneration. Specifically, the ultrasound outputted from the ultrasound output unit may facilitate cell regeneration by facilitating cell migration. More specifically, the cell migration may mean migration of normal cells or young cells. Further more specifically, the ultrasound outputted from the ultrasound output unit may facilitate cell regeneration by specifically removing senescent cells as compared to normal cells or young cells and facilitating the migration of normal cells or young cells 1.1 times or more, 1.2 times or more, 1.3 times or more, 1.4 times or more, 1.5 times or more, 1.6 times or more, 1.7 times or more, 1.8 times or more, 1.9 times or more, 2 times or more, 2.5 times or more, 3 times or more, 4 times or more, 5 times or more, 6 times or more, 7 times or more, 8 times or more, 9 times or more or 10 times or more as compared to before or without ultrasound irradiation, although not being limited thereto.

In another aspect, the present disclosure provides a kit for removing senescent cells, which includes an ultrasound output unit and an instruction, wherein the instruction describes that ultrasound outputted from the ultrasound output unit facilities phagocytosis of senescent cells by specifically stimulating the senescent cells. The ultrasound output unit, the condition of the ultrasound, the senescent cells, the stimulation, the phagocytosis, etc. are the same as described above.

In an aspect of the present disclosure, the instruction may further describe that one or more condition selected from a group consisting of the ultrasound center frequency, sound pressure, pulse repetition frequency and duty cycle of the ultrasound outputted from the ultrasound output unit is controlled and optimized depending on the type of cells to which the ultrasound is irradiated.

The kit for removing senescent cells according to an aspect of the present disclosure may further an ultrasound control unit.

In an aspect of the present disclosure, the ultrasound control unit may control and optimize one or more condition selected from a group consisting of the center frequency, sound pressure, pulse repetition frequency and duty cycle of the ultrasound depending on the subject to which the ultrasound is irradiated, the part of the subject, the type of cells or the purpose of the ultrasound irradiation.

In another aspect, the present disclosure provides a kit for promoting cell regeneration, which includes an ultrasound output unit and an instruction, wherein the instruction describes that the ultrasound outputted from the ultrasound output unit facilities phagocytosis of senescent cells and cell regeneration by specifically stimulating the senescent cells and that the cell regeneration is caused by migration of normal cells facilitated by the ultrasound outputted from the ultrasound output unit. The ultrasound output unit, the condition of the ultrasound, the senescent cells, the stimulation, the phagocytosis, etc. are the same as described above.

In an aspect of the present disclosure, the instruction may further describe that one or more condition selected from a group consisting of the ultrasound center frequency, sound pressure, pulse repetition frequency and duty cycle of the ultrasound outputted from the ultrasound output unit is controlled and optimized depending on the type of cells to which the ultrasound is irradiated.

The kit for promoting cell regeneration according to an aspect of the present disclosure may further include an ultrasound control unit.

In an aspect of the present disclosure, the ultrasound control unit may control and optimize one or more condition selected from a group consisting of the center frequency, sound pressure, pulse repetition frequency and duty cycle of the ultrasound depending on the subject to which the ultrasound is irradiated, the part of the subject, the type of cells or the purpose of the ultrasound irradiation.

In another aspect, the present disclosure provides a method for inhibiting skin aging, which includes irradiating ultrasound to skin surface, wherein the ultrasound irradiated to the skin surface facilities phagocytosis of senescent cells by specifically stimulating the senescent cells among skin cells. Specifically, the method for inhibition skin aging is a beauty care method for inhibiting skin aging. The ultrasound, the ultrasound irradiation, the cells, the senescent cells, the phagocytosis, etc. are the same as described above.

In an aspect of the present disclosure, the subject may be any subject beauty care of which is desired by inhibiting skin aging or removing senescent skin cells by irradiating ultrasound to skin, without particular limitation. Specifically, the subject may be a non-human animal such as monkey, dog, cat, rabbit, guinea pig, rat, mouse, cow, sheep, pig, goat, etc. or human, although not being limited thereto. In addition, the subject may be a subject having senescent cells. Specifically, it may be a subject wherein aging phenotypes are expressed due to insufficient removal of senescent cells, more specifically, a subject wherein the activity of senescence-associated β-galactosidase is increased, the expression of a senescent cell-specific cytokine, e.g., one or more selected from a group consisting of interleukin 6 (IL-6), interleukin 8 (IL-8), matrix metalloproteinase-1 (MMP-1) and interleukin 1β (IL-1β), is increased, or the secretion of senescence-associated secretory phenotype (SASP) is increased, further more specifically, a subject wherein skin aging has progressed, although not being limited thereto.

In another aspect, the present disclosure provides a method for specifically removing senescent cells, including irradiating ultrasound to skin surface of a subject in need of removal of senescent cells, wherein the ultrasound irradiated to the skin surface facilities phagocytosis of senescent cells by specifically stimulating the senescent cells among cells. The ultrasound, the condition of the ultrasound, the senescent cells, the stimulation, the phagocytosis, the removal of senescent cells, etc. are the same as described above.

In another aspect, the present disclosure provides a method for promoting cell regeneration, including irradiating ultrasound to skin surface of a subject in need of promotion of cell regeneration, wherein the ultrasound irradiated to the skin surface facilities phagocytosis of senescent cells and cell regeneration by specifically stimulating the senescent cells and that the cell regeneration is caused by migration of cells facilitated by the ultrasound. The ultrasound, the condition of the ultrasound, the senescent cells, the stimulation, the phagocytosis, the promotion of cell regeneration, the migration of cells, etc. are the same as described above.

Hereinafter, the constitution and effect of the present disclosure are described more specifically through an example and test examples. However, the following example and test examples are provided only to help understanding of the present disclosure and the category and scope of the present disclosure are not limited by them.

### [Example] Stimulation of Cells Using Ultrasound

In order to investigate whether ultrasound irradiation removes senescent cells by specifically stimulating the senescent cells, experiment was conducted as follows using human dermal fibroblasts HS68 (CRL-1635) purchased form ATCC (USA).

First, the HS68 cells were cultured in DMEM medium supplemented with 10% fetal bovine serum (FBS). The medium and medium supplement were purchased from Gibco. In the test examples described below, the HS68 cells that were subcultured within 20 passages (P20), mainly P16-20, were used as young cells (young) of the control group, and the HS68 cells that were subcultured for about 40 passages, mainly P38-43, as senescent cells as a result of prolonged cell division, were used as senescent cells (old) of the test group.

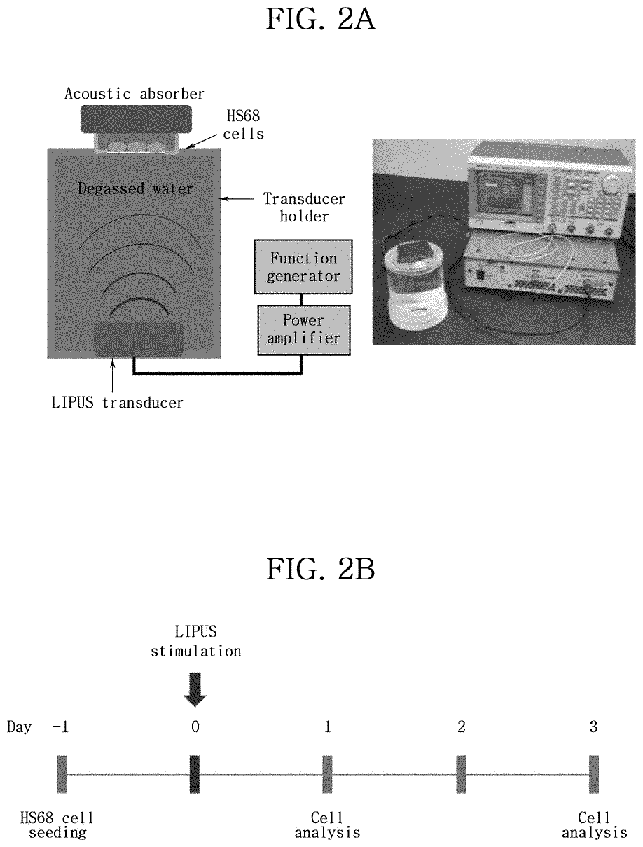

Ultrasound irradiation was performed as shown in FIG. 2A. After placing a cell culture dish with a diameter of 35 mm on a vessel (SPL Life Sciences) equipped with a transducer (NMB-M155A, Dong II Technology) for low-intensity ultrasound stimulation, ultrasound stimulation was induced. Specifically, 1×10⁵ of young cells or senescent cells were cultured on a glass bottom confocal dish (35 pi dish) and the next day ultrasound was applied to the cells. The thickness of confocal dish was 180 μm, so that ultrasound could easily pass through. Specifically, the ultrasound condition for stimulation of cells was as follows: 1.5 MHz, 243 kPa sound pressure, 20% duty cycle, 100 Hz repetition rate, 800 mW/cm² intensity, one irradiation for 20 minutes. At day 2 (one day after the ultrasound irradiation) and day 4 (3 days after the ultrasound irradiation) cells were collected for the biological experiments. The experimental schedule is illustrated in FIG. 2B.

### [Test Example 1] Cell Viability, Cell Proliferation and Cell Cycle Analysis after Ultrasound Irradiation

Experiments were performed as follows in order to investigate whether cell viability, cell proliferation and cell cycle are changed by ultrasound irradiation.

### [Test Example 1-1] Cell Viability Measurement

Trypan blue exclusion test was performed as follows in order to investigate the change in cell viability by ultrasound irradiation.

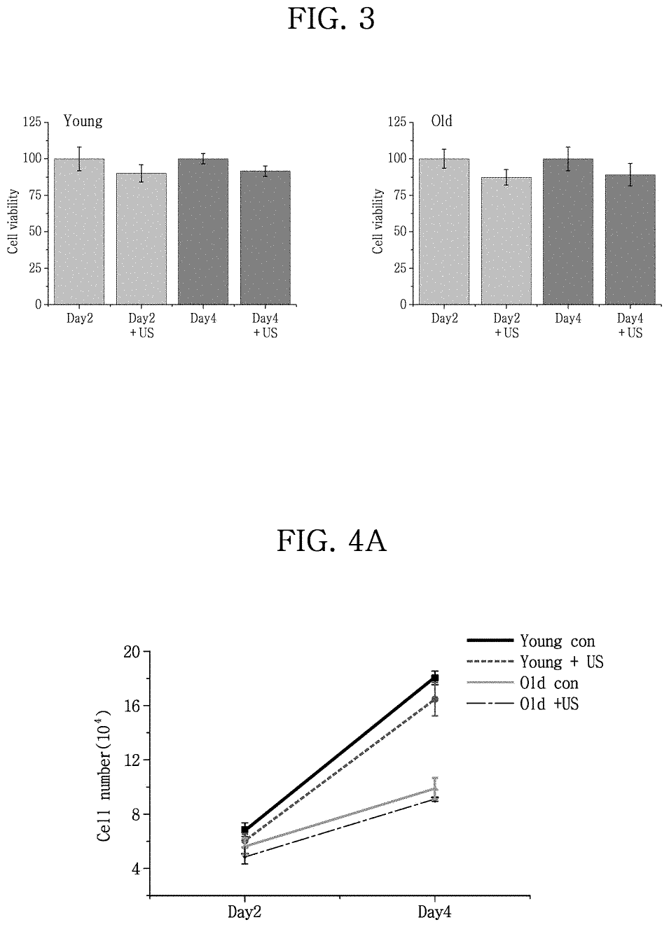

First, cells at day 2 and day 4 illustrated in FIG. 2B were washed with the PBS and detached using Trypsin EDTA (Gibco), then mixed with Trypan blue (Sigma-Aldrich) at a ratio of 1:1. The mixed cell solution was added to a hemocytometer (DHC-N01-5, InCyto) and the number of living cells not stained with Trypan blue was counted. The change in cell viability was measured as a function of ultrasound intensity starting from 0 to 7.5, 60 and 800 mW/cm². The change in cell viability with the ultrasound intensity of 800 mW/cm² is shown in FIG. 3A and FIG. 3B. The viability of control cells without ultrasound irradiation was set to 1, and the relative cell viability after ultrasound irradiation was shown in FIG. 3A and FIG. 3B As shown in FIG. 3A and FIG. 3B, both the young cells and the senescent cells showed slight decrease in cell viability 1 day (day 2+US) or 3 days (day 4+US) after the ultrasound irradiation, but the change was not statistically significant. Therefore, it was confirmed that the change in cell viability by ultrasound irradiation is insignificant.

### [Test Example 1-2] Cell Proliferation Measurement

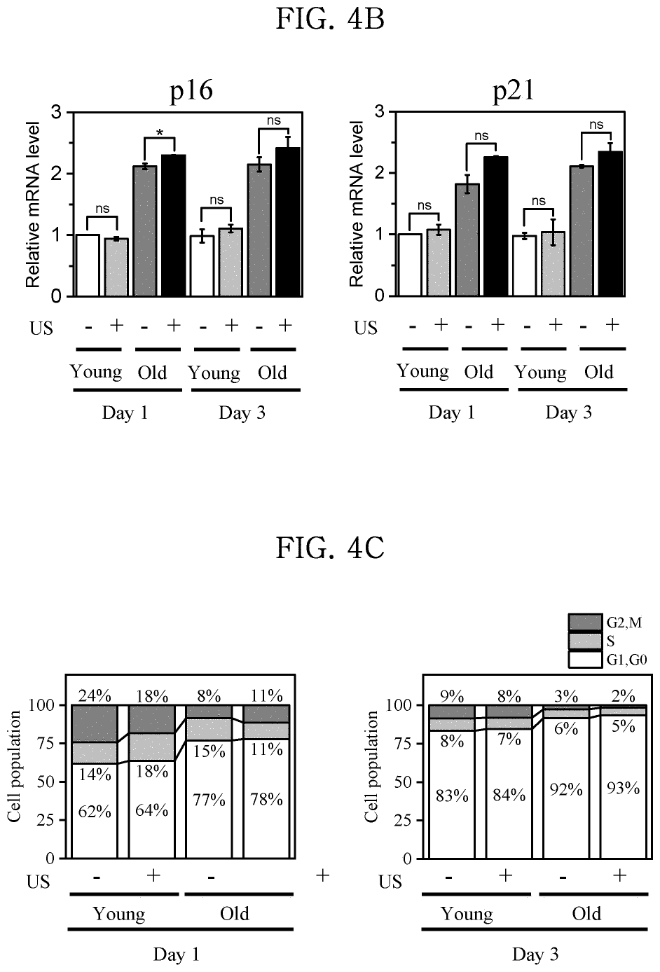

In order to investigate whether cell proliferation was affected by ultrasound irradiation, cell proliferation rate was measured from the experiment of Test Example 1-1. The result is shown in FIG. 4A. The proliferation rate of the senescent cells is two times slower than that of young cells. However, both the young cells and the senescent cells showed no change in cell proliferation rate (the slope of graphs in FIG. 4A) after the ultrasound irradiation.

### [Test Example 1-3] Cell Cycle Analysis

In order to investigate the change in cell cycle by ultrasound irradiation, the mRNA expression levels of proteins involved in cell cycle, specifically p16 and p21, were measured by qRT-PCR as described below. The change in the expression level of the mRNAs of the proteins was investigated 3 days after the ultrasound irradiation (4 days after the subculturing).

First, RNAs were isolated from the cells of Example using a GeneJET RNA purification kit (Thermo Fisher) 4 days after the subculturing. Then, cDNAs were synthesized from 1 μg of the RNAs and subjected to target gene expression analysis using SYBR Green. The result is shown in FIG. 4B. The primers described in Table 1 were used.

TABLE 1

| Target | | | |
|---|---|---|---|
| gene | Primers | Base sequence | SEQ ID NO |
| p16 | Forward primer | 5′-GATCCAGGTGGGTAGAAGGTC-3′ | SEQ ID NO 1 |
| | Reverse primer | 5′-CCCCTGCAAACTTCGTCCT-3′ | SEQ ID NO 2 |
| p21 | Forward primer | 5′-AGGTGGACCTGGAGACTCTCAG-3′ | SEQ ID NO 3 |
| | Reverse primer | 5′-TCCTCTTGGAGAAGATCAGCCG-3′ | SEQ ID NO 4 |
| GAPDH | Forward primer | 5′-ACCCACTCCTCCACCTTTGA-3′ | SEQ ID NO 5 |
| | Reverse primer | 5′-CTGTTGCTGTAGCCAAATTCGT-3′ | SEQ ID NO 6 |

In addition, cell cycle was measured using the cells that had been subcultured for 4 days by FACS analysis. Specifically, the cells of Example 4 days after the subculturing were isolated as single cells using Trypsin EDTA and then washed with cold PBS. Then, after fixing treated cells using a 70% ethanol solution, DNA was stained using a 50 μg/mL propidium Iodide (PI) solution at 4° C. for 10 minutes. The quantity of DNA stained with Pl was investigated by flow cytometry (CytoFLEX, Beckman). The data were analyzed using the FlowJo program. The result is shown in FIG. 4C.

As shown in FIG. 4B, compared with the young cell, the expressions of both p16 and p21 in senescent cells was increased, but these were hardly affected by the ultrasound.

Also, as shown in FIG. 4C, the distribution of cells cycle arrested in G0/G1 phases were increased in the senescent cells (old) as compared to the young cells (young). When the ultrasound was irradiated, although the distribution of cells in G2 phase was changed in both the young cells (young, US) and the senescent cells (old, US), the change was insignificant when considering the change in cell cycle of the young cells and the senescent cells.

From FIG. 4B and FIG. 4C, it was confirmed cell cycles were not affected by ultrasound irradiation.

From the results of Test Examples 1-1 to 1-3, it was confirmed that the ultrasound irradiation does not affect cell viability, cell proliferation rate or cell cycle in both the young cells and the senescent cells.

### [Test Example 2] Senescence-Associated β-Galactosidase Activity Assay

In order to examine whether ultrasound irradiation changes senescence-associated phenotypes, the activity of SA β-gal protein was measured by X-gal staining using a β-gal staining kit (Cell Signaling, S9860). The SA β-gal is a protein known to be increased in senescent cells.

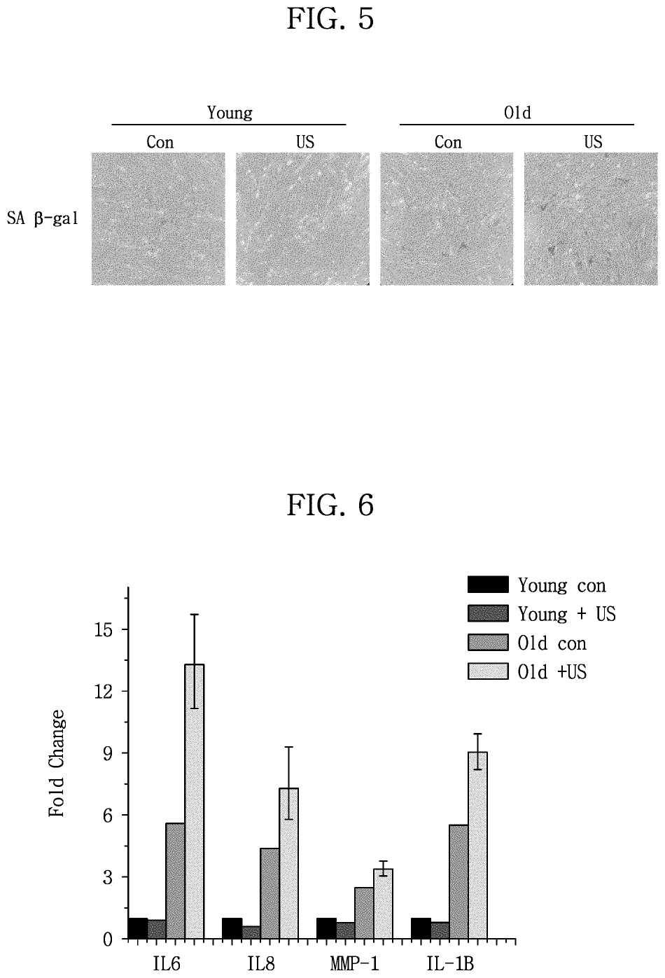

The cells of Example 2 days and 4 days after the subculturing were fixed and β-gal solution (1 mg/mL) was added. Cells in culture dish were sealed and placed in the incubator at 37° C. overnight. Then treated cells were washed with PBS, and color change was observed using an optical microscope. The result is shown in FIG. 5.

As shown in FIG. 5, the quantity and activity of the SA β-gal protein in senescent cells (old) was higher as compared to young cells (young) before ultrasound irradiation (con), resulting in a higher amount of blue staining. Whereas no significant change was observed for the young cells 3 days after the ultrasound irradiation (4 days after the subculturing, US), the senescent cells showed enhanced activity of SA β-gal.

### [Test Example 3] SASP Measurement

In order to examine whether ultrasound irradiation changes senescence-associated phenotypes, SASP was measured by qRT-PCR in the same manner as in Test Example 1-3.

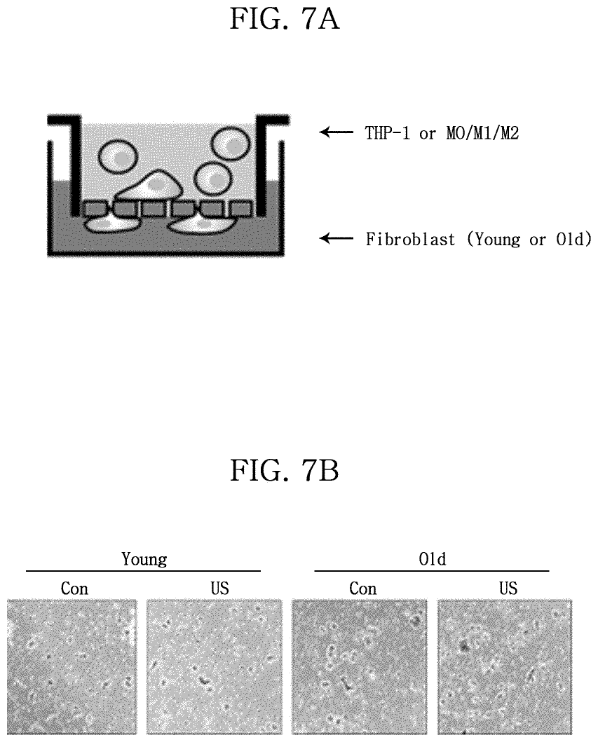

The primers described in Table 2 were used, and the result of qRT-PCR is shown in FIG. 6.

TABLE 2

| Target | | | |
|---|---|---|---|
| genes | Primers | Base sequence | SEQ ID NO |
| IL-6 | Forward primer | 5′-CCACACAGACAGCCACTCACC-3′ | SEQ ID NO 7 |
| | Reverse primer | 5′-CTACATTTGCCGAAGAGCCCTC-3′ | SEQ ID NO 8 |
| IL-8 | Forward primer | 5′-CTCTCTTGGCAGCCTTCCTGATT-3′ | SEQ ID NO 9 |
| | Reverse primer | 5′-AACTTCTCCACAACCCTCTGCAC-3′ | SEQ ID NO 10 |
| MMP-1 | Forward primer | 5′-AGCTAGCTCAGGATGACATTGATG-3′ | SEQ ID NO 11 |
| | Reverse primer | 5′-GCCGATGGGCTGGACAG-3′ | SEQ ID NO 12 |
| IL-1B | Forward primer | 5′-CAGCTACGAATCTCCGACCAC-3′ | SEQ ID NO 13 |
| | Reverse primer | 5′-GGCAGGGAACCAGCATCTTC-3′ | SEQ ID NO 14 |

As shown in FIG. 6, the SASP cytokines were increased in the senescent cells (old) as compared to the young cells (young). The change in the SASP cytokines was insignificant in young cells 3 days after the ultrasound irradiation (4 days after the subculturing, young+US). However, the SASP cytokines in senescent cells were significantly increased.

From Test Examples 2 and 3, it was confirmed that the ultrasound stimulation has no effect SA β-gal and SASP on young cells, but increases both SA β-gal and SASP in the senescent cells.

### [Test Example 4] Investigation of Facilitation of Senescent Cell-Specific Phagocytosis by Ultrasound Irradiation

In order to investigate whether ultrasound irradiation facilitates immune cell recruiting by secretion of SASP in senescent cells, migration rate of monocytes and macrophages measured.

### [Test Example 4-1] Cell Migration Rate of Monocytes and Macrophages

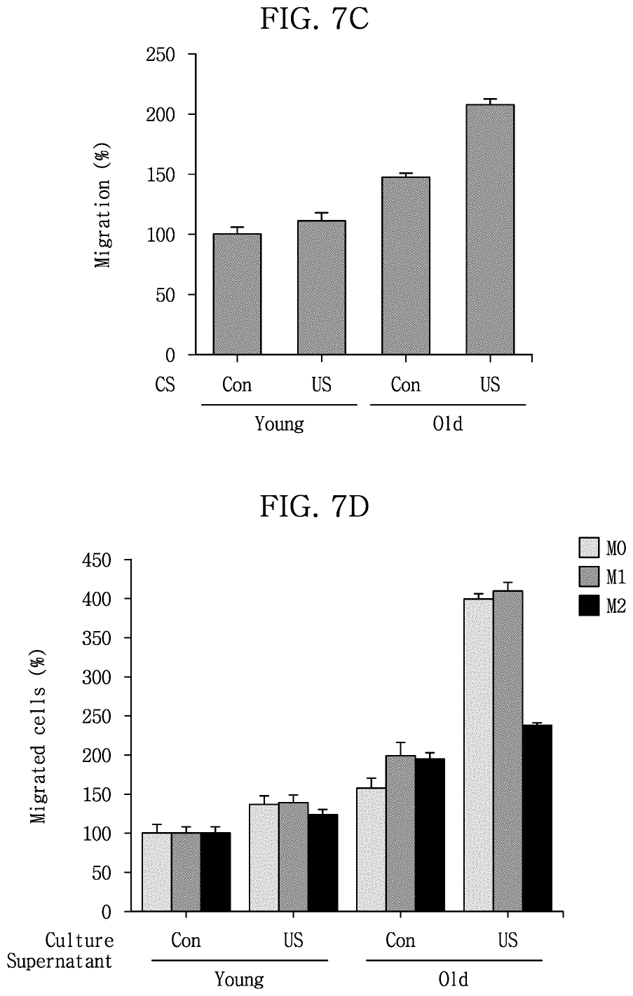

The effects of ultrasound irradiation on immune cell responses was determined by transwell migration assay. Human monocytes THP-1 were acquired from Korea Cell Line Bank (Seoul, Korea), and transwell inserts with a pore size of 8 μm (Corning Co., NY, USA) and were used. As shown in FIG. 7A, monocytes or macrophages were cultured on the upper side of a transwell, and young cells or senescent cells irradiated or not irradiated with ultrasound were cultured on the lower side of the transwell. Due to the cytokines secreted by the young cells or the senescent cells in the supernatant, the monocytes or the macrophages migrate toward the lower side of the transwell, and the number of migrated cells was measured by crystal violet staining.

Specifically, THP-1 cells were cultured in RPMI-1640 medium (Welgene, Korea) supplemented with 10% fetal bovine serum (FBS), 1% penicillin/streptomycin (P/S) and 0.1 mM β-mercaptoethanol under the condition of 37° C. and 5% CO₂. In order to differentiate them into macrophages, the cells were treated with phorbol 12-myristate 13-acetate (PMA). Briefly, THP-1 monocytes were differentiated into M0 macrophages by treating with 20 nM PMA (Tocris Bioscience) for 2 days. Then, the M0 macrophages were polarized to M1 macrophages by treating with 20 ng/mL LPS and 20 ng/mL INF-γ or to M2 macrophages by treating with 40 ng/mL IL-4 and 20 ng/mL IL-13.

Then, the 2×10⁵ of monocytes (THP-1) and macrophages (M0 macrophages, M1 macrophages or M2 macrophages) were diluted in 200 μL of RPMI-1640 (Welgene, Korea) and added to an insert (upper well). 800 μL of culture media from the young cells or the senescent cells of Example not irradiated with ultrasound or irradiated with ultrasound for 3 days (4 days after the subculturing) were added to lower wells. Then, the insert was placed with the lower well with the culture media, and incubated for 3 hours at 37° C. with 5% CO₂. Then, the cells at the top portion of filter, which did not migrate and infiltrate, were removed with a swab and the cells that migrated through the polycarbonate filter were stained with crystal violet. The result is shown in FIGS. 7B-7D.

FIG. 7A shows the schematics of transwell assay. FIG. 7B shows optical images of monocytes migrated from the upper well after staining them with crystal violet. As shown in FIG. 7B, the culture media from senescent cells promoted migration of monocytes compared to that from young cells. Furthermore, THP migration was highest when incubated with culture media from ultrasound irradiated senescent cells. Through this, it was confirmed that the SASP enhanced by ultrasound irradiation in senescent cell specifically facilitates the migration of monocytes and macrophages.

FIG. 7C shows a result of the same experiment with three types of macrophages (M0, M1 and M2) instead of the monocytes (THP-1). The change in the migration of the three types of macrophages was not significant with young cells regardless of ultrasound irradiation, the migration of M0 macrophages and M1 macrophages, which are important in phagocytosis, was significantly increased with the culture media from senescent cells after ultrasound irradiation. But, the increase in the migration of M2 macrophages was insignificant.

### [Test Example 4-2] Measurement of SASP Involved in Immune Cell-Recruiting

It was confirmed from Test Example 4-1 that the migration of monocytes and macrophages is promoted by senescent cell after ultrasound irradiation. In order to investigate whether senescent cells increase SASP secretion involved in immune cell recruiting, mRNAs levels of cytokines known to recruit macrophages or immune cells, were measured by qRT-PCR in the same manner as in Test Example 1-3.

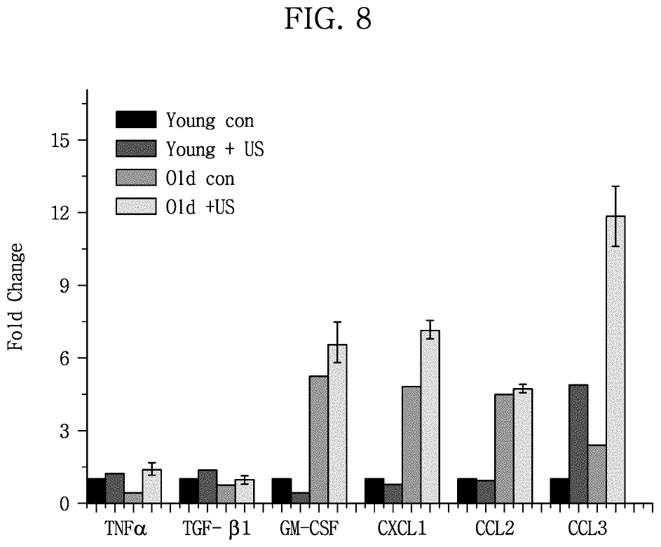

The primers described in Table 3 were used, and the result of qRT-PCR is shown in FIG. 8.

TABLE 3
Target

| genes | Primers | Base sequence | SEQ ID NO |
|---|---|---|---|
| TNF-α | Forward primer | 5′-ATGAGCACTGAAAGCATGATCC-3′ | SEQ ID NO 15 |
| | Reverse primer | 5′-GAGGGCTGATTAGAGAGAGGTC-3′ | SEQ ID NO 16 |
| TGF-β1 | Forward primer | 5′-AGGGCTACCATGCCAACTTCT-3′ | SEQ ID NO 17 |
| | Reverse primer | 5′-CCGGGTTATGCTGGTTGTACA-3′ | SEQ ID NO 18 |
| GM- | Forward primer | 5′-CACTGCTGCTGAGATGAATGAAA-3′ | SEQ ID NO 19 |
| CSF | Reverse primer | 5′-GTCTGTAGGCAGGTCGGCTC-3′ | SEQ ID NO 20 |
| CXCL1 | Forward primer | 5′-GAAAGCTTGCCTCAATCCTG-3′ | SEQ ID NO 21 |
| | Reverse primer | 5′-CTTCCTCCTCCCTTCTGGTC-3′ | SEQ ID NO 22 |
| CCL2 | Forward primer | 5′-GAGAGGCTGAGACTAACCCAGA-3 | SEQ ID NO 23 |
| | Reverse primer | 5′-ATCACAGCTTCTTTGGGACACT-3′ | SEQ ID NO 24 |
| CCL3 | Forward primer | 5′-GGCTCTCTGCAACCAGTTCT-3′ | SEQ ID NO 25 |
| | Reverse primer | 5′-TGAAATTCTGTGGAATCTGCC-3′ | SEQ ID NO 26 |

As shown in FIG. 8, the change in the inflammatory cytokines of TNF-α and TGF-β by ultrasound irradiation was insignificant regardless of the cell type. However, the senescent cells (old con) showed increase in CSF, CXCL1, CCL2 and CCL3, which are known as immune cell-recruiting cytokines, and the three cytokines were further increased upon ultrasound irradiation.

From Test Examples 4-1 and 4-2, it was confirmed that immune cell-recruiting cytokines are specifically increased by ultrasound irradiation only in senescent cells and, consequently, the migration of monocytes or macrophages (M0 macrophages and M1 macrophages) is increased afterwards.

### [Test Example 5] Wound Healing Assay

In order to investigate whether cell regeneration is facilitated by ultrasound irradiation through increased secretion of SASP by senescent cells, wound healing assay was performed. First, normal fibroblast was cultured and some specific area of the fibroblast cells were scratched out to mimic the damaged tissue. Then, the culture media from young cells or senescent cells that were non-treated or ultrasound irradiated was collected and treated to the normal fibroblast with scratch.

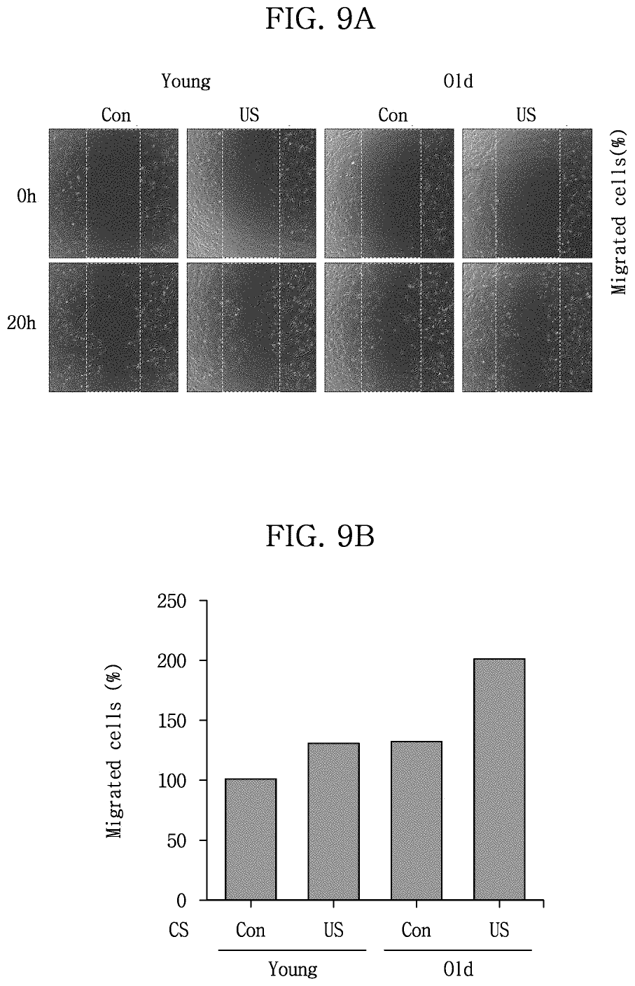

Specifically, fibroblasts of a single layer were seeded onto a 24-well plate to 90% or higher confluence. Then, the bottom of the plate was scratched using a yellow pipette tip to make a part where the cells were not attached. Then, after removing the detached cells by washing twice with PBS, the plate was treated with a culture media of young cells or senescent cells of Example not irradiated with ultrasound or a culture media of young cells or senescent cells irradiated with ultrasound for 3 days (4 days after the subculturing). The area where the scratch was made imaged with a microscope (×40) at 0 hour and 20 hours after the wound was formed, and the decrease in the area for 20 hours was measured with ImageJ (National Institutes of Health, USA). The result is shown in FIGS. 9A and 9B.

As shown in FIG. 9A, whereas the young cells (young) did not show significant migration of normal fibroblasts by the ultrasound irradiation, the treatment with a culture media of the senescent cells (old) irradiated with ultrasound resulted in the highest degree of migration of normal fibroblasts. That is to say, the change in senescent cell-specific cytokines by ultrasound irradiation facilitates the migration of normal fibroblast, and is effective in facilitating cell regeneration after removal of cells by phagocytosis.

In conclusion, it was confirmed that ultrasound irradiation selectively stimulates senescent cells and promotes secretion of SASP factors, leading to enhanced immune cell migration. Consequently, elimination of senescent cells, phagocytosis can be enhanced by monocytes and macrophages (M0 macrophages and M1 macrophages). Also, the regeneration ability is increased due to facilitated migration of normal fibroblasts to the damaged region. Through this, it can be demonstrated that ultrasound has a superior effect of removing senescent cells and facilitating cell regeneration by specifically stimulating senescent cells, thereby facilitating phagocytosis specifically in the senescent cells without affecting cell viability, cell proliferation rate and cell cycle.

1. A device for removing senescent cells, comprising an ultrasound output unit, wherein ultrasound outputted from the ultrasound output unit facilities phagocytosis of senescent cells by specifically stimulating the senescent cells.

2. The device for removing senescent cells according to claim 1, wherein the ultrasound outputted from the ultrasound output unit has one or more characteristic selected from a group of consisting of a center frequency of 5 kHz to 450 MHz, a sound pressure of −10 MPa to 10 MPa, a pulse repetition frequency of 0.02 Hz to 500 kHz and a duty cycle of 0.1-99.9%.

3. The device for removing senescent cells according to claim 1, wherein the senescent cell is one or more selected from a group consisting of a senescent fibroblast, a senescent melanocyte, a senescent keratinocyte, a senescent muscle cell, a senescent epithelial cell, a senescent vascular endothelial cell, a senescent bone cell, a senescent cartilage cell, a senescent cardiac muscle cell, a senescent liver cell, a senescent pancreatic cell and a senescent kidney cell.

4. The device for removing senescent cells according to claim 1, wherein the ultrasound outputted from the ultrasound output unit senescent cell-specifically increases the activity of senescence-associated β-galactosidase, or the expression of one or more senescent cell-specific cytokine selected from a group consisting of interleukin 6 (IL-6), interleukin 8 (IL-8), matrix metalloproteinase-1 (MMP-1) and interleukin 1β (IL-1β).

5. The device for removing senescent cells according to claim 1, wherein the ultrasound outputted from the ultrasound output unit specifically facilitates the migration of monocytes or macrophages in senescent cells.

6. The device for removing senescent cells according to claim 5, wherein the macrophages are M0 macrophages or M1 macrophages.

7. The device for removing senescent cells according to claim 1, wherein the ultrasound outputted from the ultrasound output unit senescent cell-specifically increases the expression of one or more immune cell-recruiting cytokine selected from a group consisting of colony-stimulating factor (CSF), chemokine (C-X-C motif) ligand 1 (CXCL1) and chemokine (C-C motif) ligand 3 (CCL3).

8. The device for removing senescent cells according to claim 1, wherein the ultrasound outputted from the ultrasound output unit has no senescent cell-specific effect on one or more selected from a group consisting of cell viability, cell proliferation and cell cycle.

9. The device for removing senescent cells according to claim 1, wherein the ultrasound outputted from the ultrasound output unit specifically facilitates the death of senescent cells.

10. The device for removing senescent cells according to claim 1, wherein the ultrasound outputted from the ultrasound output unit facilitates cell regeneration.

11. The device for removing senescent cells according to claim 10, wherein the facilitation of cell regeneration is a facilitation of cell migration by the ultrasound outputted from the ultrasound output unit.

12. A kit for removing senescent cells, comprising an ultrasound output unit and an instruction, wherein the instruction describes that ultrasound outputted from the ultrasound output unit facilities phagocytosis of senescent cells by specifically stimulating the senescent cells.

13. The kit for removing senescent cells according to claim 12, wherein the instruction further describes that one or more condition selected from a group consisting of the ultrasound center frequency, sound pressure, pulse repetition frequency and duty cycle of the ultrasound outputted from the ultrasound output unit is controlled depending on the type of cells to which the ultrasound is irradiated.

14. The kit for removing senescent cells according to claim 12, wherein the ultrasound outputted from the ultrasound output unit is low-intensity ultrasound having a center frequency of 5 kHz to 450 MHz.

15. The kit for removing senescent cells according to claim 12, wherein the senescent cell is one or more selected from a group consisting of a senescent fibroblast, a senescent melanocyte, a senescent keratinocyte, a senescent muscle cell, a senescent epithelial cell, a senescent vascular endothelial cell, a senescent bone cell, a senescent cartilage cell, a senescent cardiac muscle cell, a senescent liver cell, a senescent pancreatic cell and a senescent kidney cell.

16. A method for specifically removing senescent cells, comprising irradiating ultrasound to skin surface of a subject in need of removal of senescent cells, wherein the ultrasound irradiated to the skin surface facilities phagocytosis of senescent cells by specifically stimulating the senescent cells among cells.

17. The method for specifically removing senescent cells according to claim 16, wherein the ultrasound is low-intensity ultrasound having a center frequency of 5 kHz to 450 MHz.

18. The method for specifically removing senescent cells according to claim 16, wherein the cell is a skin cell and the skin cell is one or more selected from a group consisting of a fibroblast, a melanocyte and a keratinocyte.

19. The method for specifically removing senescent cells according to claim 16, wherein the ultrasound has one or more characteristic selected from a group of consisting of a center frequency of 5 kHz to 450 MHz, a sound pressure of −10 MPa to 10 MPa, a pulse repetition frequency of 0.02 Hz to 500 kHz and a duty cycle of 0.1-99.9%.

20. The method for specifically removing senescent cells according to claim 16, wherein the ultrasound irradiated to the skin surface has at least one of the following characteristics:
i) the ultrasound senescent cell-specifically increases the activity of senescence-associated β-galactosidase, or the expression of one or more senescent cell-specific cytokine selected from a group consisting of interleukin 6 (IL-6), interleukin 8 (IL-8), matrix metalloproteinase-1 (MMP-1) and interleukin 1β (IL-1β);
ii) the ultrasound specifically facilitates the migration of monocytes or macrophages in senescent cells;
iii) the ultrasound senescent cell-specifically increases the expression of one or more immune cell-recruiting cytokine selected from a group consisting of colony-stimulating factor (CSF), chemokine (C-X-C motif) ligand 1 (CXCL1) and chemokine (C-C motif) ligand 3 (CCL3);
iv) the ultrasound has no senescent cell-specific effect on one or more selected from a group consisting of cell viability, cell proliferation and cell cycle;
v) the ultrasound specifically facilitates the death of senescent cells; and
vi) the ultrasound outputted from the ultrasound output unit facilitates cell regeneration.
